(19) United States
(12) Patent Application Publication
(10) Pub. No.: US 2015/0190161 A1
(43) Pub. Date: Jul. 9, 2015

(54) Cutter for Use in Surgery

(71) Applicants: Albert Barokas, Geneva (CH); Bruno Damascalli, Milano (IT)

(72) Inventors: Albert Barokas, Geneva (CH); Bruno Damascalli, Milano (IT)

(21) Appl. No.: 14/421,857

(22) PCT Filed: Jul. 30, 2013
(86) PCT No.: PCT/IB2013/056251

§ 371 Date: Feb. 16, 2015

(30) Foreign Application Priority Data
Aug. 14, 2012 (IB) PCT/IB2012/054129

(51) Int. Cl. A61B 17/32; A61B 17/34; A61B 17/3211

(52) U.S. Cl. CPC A61B 17/32002; A61B 17/3211; A61B 17/34

(57) ABSTRACT

A cutter (10) for use in surgery, comprising a needle (20) having a cutting end (21) and a cursor (30) slidably mountable on or mounted on the needle (20). The cursor has a cursor housing (32) slidably mountable on the needle (20) with the cursor housing (32) having an open end facing the needle cutting end, and a pair of flat cutter blades (15) mounted in the cursor housing (32) on either side of the needle (20). The cutter blades are mounted for movement together relative to the cursor housing (32) between a first stored position wherein the cutter blades (15) are enclosed in the cursor housing (32) and a second projecting position wherein cutting edges (16) of the cutter blades (15) project from the open end of the cursor housing (32). The cutter blades (15) are movable from the first to the second position against the action of biasing means (38) that act to return the cutter blades to the first position.

### FIELD OF THE INVENTION

This invention relates to a cutter which is suitable in particular for use in surgery, as well as a cursor and cutting blade assembly for use in such a cutter.

### BACKGROUND OF THE INVENTION

Surgical interventions frequently involve the cutting of an incision in a patient's skin. In one type of operation, the surgeon places a needle where the incision is to be made, then by means of a scalpel manually makes an incision on ether side of the needle. However, this requires great dexterity and it is difficult to make a precise incision. Accidents can occur if the incision is made too deep or too long as the surgeon does not have good enough control.

Such operations can be part of biopsy, drainage or angiography. Incisions can also be made for the purpose of creating a cavity to introduce an implantable device, for example by first making an incision, then introducing an instrument such as the blunt dissector described in WO 2012/059870 to create the cavity.

### SUMMARY OF THE INVENTION

An object of the invention is to provide a cutter for use in surgery with which a surgeon has greater control in making an incision and therefore can make a more precise incision with less risk of accident.

The invention therefore provides a cutter for use in surgery, comprising a needle having a cutting end, and a cursor slidably mountable on or mounted on the needle. This cursor comprises a cursor housing slidably mountable on or mounted on the needle with the cursor housing having an open end facing the needle cutting end; and a pair of flat cutter blades mounted in the cursor housing so as to be disposed on either side of the needle when the cursor is mounted on the needle. The cutter blades have cutting edges facing the open end of the cursor housing, the cutter blades being mounted for movement together relative to the cursor housing between a first stored position wherein the cutter blades are enclosed in the cursor housing and a second projecting position wherein the cutting edges of the cutter blades project from the open end of the cursor housing. The cutter blades are movable from the first to the second position against the action of biasing means that act to return the cutter blades to the first position.

The inventive cutter is operable for making a cut or an incision in a surface by bringing the needle cutting end into contact with the surface to be cut, sliding the cursor along the needle up to adjacent surface to be cut, and moving the cutter blades to the projecting second position against the action of the biasing means to bring the cutting edges to a cutting position for making a cut/incision in the surface on either side of the needle.

The cutter blades are usually disposed in the cursor housing symmetrically about the needle, for example wherein the cutting edges of the cutting blades each make an angle of at most 45 degrees and preferably up to 30 degrees with the needle.

The cursor housing is usually substantially flat such that the cutter blades lie flat in the substantially flat housing.

Advantageously, the cursor housing has, at an end of the cursor opposite its open end, two laterally-projecting finger grips for facilitating manipulation of the cursor housing. The cutter moreover preferably comprises an actuator that projects from the cursor housing for facilitating movement of the cutter blades from the first to the second position. Such actuator can project from a flat face of the cursor housing, in which case it acts like a slide, or the actuator can be a press button typically located on a top edge of the cursor housing, opposite to its open end from which the cutting blades can be made to project.

The needle can be pre-assembled with the cursor housing, forming a pre-assembled unit with the cursor housing slidably mounted on the needle. Alternatively, the needle and cursor housing can be delivered separately as a set of parts, in which case the user/surgeon assembles the cursor housing on the needle in preparation for use.

The needle can be hollow in which case the cutter can be used in combination with a flexible wire or tube that can be passed through the needle.

The needle typically has a diameter in the range 1 mm to 6 mm depending on the envisaged uses. Each cursor housing will be adapted to the size of needle it is designed to accommodate, so the cursor housing has a through-passage of corresponding dimensions for slidably receiving the needle.

The cutter blades can be assembled with a central cylindrical piece or part-cylindrical piece to form a cutter blade assembly having one cutter blade on either side of the central cylindrical or part-cylindrical piece, like a delta wing for example.

The cutter blades are conveniently mounted on a support slidably mounted in the cursor housing. Is such case, the biasing means such as a spring or an elastic band, can act on the slidable needle-support.

A further aspect of the invention is a cursor and cutting blade assembly of a cutter as discussed above, which cutter is adapted to be used in combination with a needle having a cutting end. In this case, the cursor and cutting blade assembly comprise a cursor housing slidably mountable on an associated needle, the cursor housing when mounted on the needle having an open end facing the needle cutting end; and a pair of flat cutter blades mounted in the cursor housing on either side of the needle when mounted, the cutter blades having cutting edges facing the open end of the cursor housing, the cutter blades being mounted for movement together relative to the cursor housing between a first stored position wherein the cutter blades are enclosed in the cursor housing and a second projecting position wherein the cutting edges of the cutter blades project from the open end of the cursor housing, the cutter blades being movable from the first to the second position against the action of biasing means that act to return the cutter blades to the first position. In this assembly, the cursor housing will have a central through-passage passing between the cutting blades, for slidably receiving a needle of corresponding dimensions.

### BRIEF DESCRIPTION OF DRAWINGS

The invention will be further described by way of example with reference to the drawings, wherein:

FIG. 1 is a schematic elevational view of a cutter according to the invention showing its inner structure, with the cutter in a closed position in which the cutter blades are retracted in the cursor housing.

FIG. 2 is a front view and a side view of a cutter blade assembly.

FIG. 3 is a schematic cross-section through the cutter in the position of FIG. 1 at the level of the cutting blades.

FIG. 4 1 is a schematic elevational view of the cutter of FIG. 1, with the cutter in an open position in which the cutter blades project for making an incision in the skin.

### DETAILED DESCRIPTION

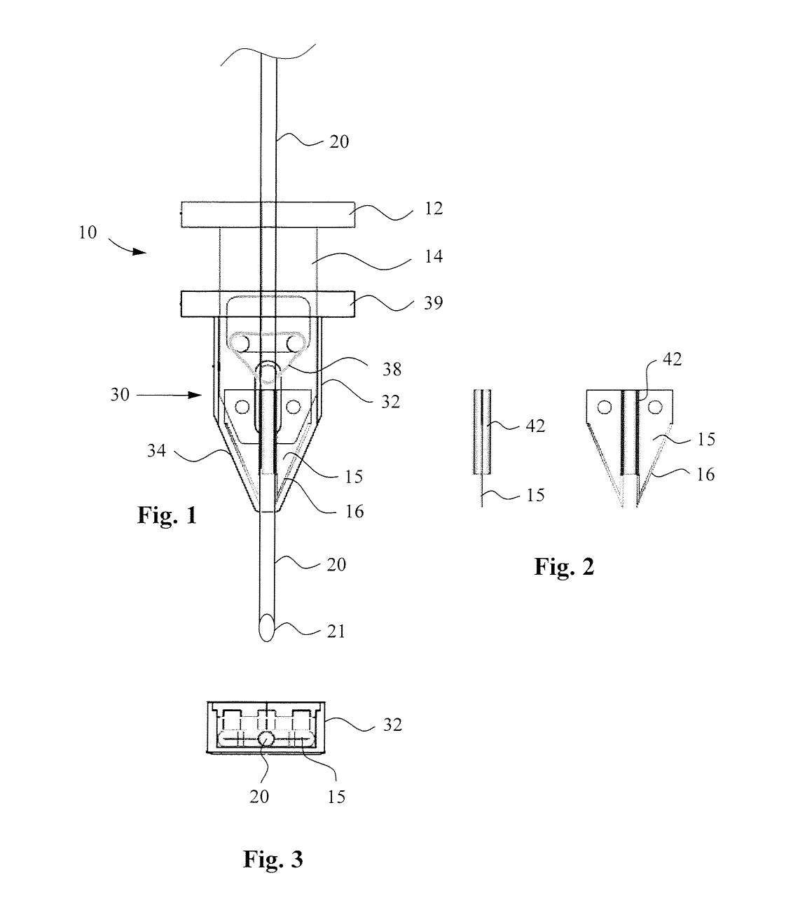

With reference to FIG. 1, a cutter 10 according to the invention comprises a hollow needle 20 having a cutting end 21; and a cursor 30 slidably mountable on or, as shown, actually mounted on the needle 20, so the cursor 30 can be slid along the needle 20. For this purpose, the cursor 30 has a central through-passage of appropriate dimensions for receiving the needle 20.

The cursor 30 comprises: a cursor housing 32 slidably mounted on the needle with the cursor housing having an open end 34 facing the needle cutting end 21. In the cursor housing 32 is slidably mounted a cutter holder 14 carrying on its forward end a pair of flat cutter blades 15 mounted on either side of the needle 20. The cutter blades 15 together form scalpel mounted as a cutter-blade unit on the holder 14 that is slidably mounted in the cursor 30.

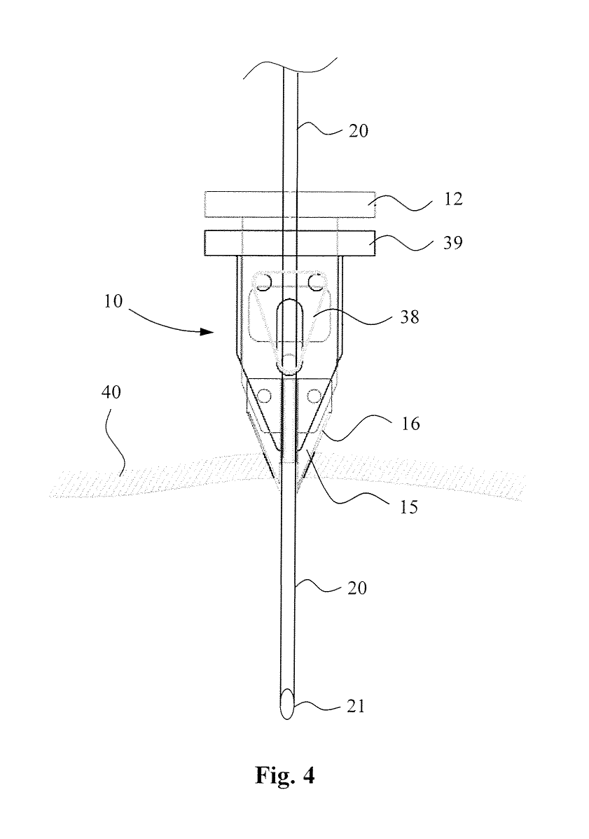

The cutter blades 15 have cutting edges 16 facing the open end of the cursor housing 32. As shown, the open end 34 of cursor housing 32 is tapered towards the end. In its sides it has slits for the passage of the cutting edges 16 of the cutter blades 15. The cutter blades 15 are mounted for movement together relative to the cursor housing 32 between a first stored position (FIG. 1) wherein the cutter blades 15 are enclosed in the cursor housing 32 and a second projecting position (FIG. 4) wherein the cutting edges 16 of the cutter blades 15 project from the open end of the cursor housing 32. The cutter blades 15 are movable from the first to the second position against the action of biasing means that act to return the cutter blades 15 to the first position.

In this example, the biasing means is an elastic band 38 made of rubber, that passes around embossments on the cutter-blade unit and in the cursor housing 32.

As shown, the cursor housing 32 advantageously has, at the end of the cursor 30 opposite is tapered end 34, two laterally-projecting finger grips 39 for facilitating manipulation of the cursor housing 32. Furthermore the illustrated cutter 10 comprises an actuator 12 for example a press button that projects from the cursor housing 32 on the holder 14 of the slidable cutter-blade unit, for facilitating movement of the cutter blades 15 from the first to the second position.

The described cutter 10 is operable for making a cut or an incision in a surface 40, namely a patient's skin, by bringing the needle cutting end 21 into contact with the surface to be cut, pressing the needle 20 in, sliding the cursor 30 along the needle 20 up to adjacent the skin 40 and moving the cutter blades 15 to the projecting second position as shown in FIG. 4 against the action of the elastic band 38 by pressing in the actuator 12 to bring the cutting edges 16 to a cutting position for making a cut or incision in the surface 40 on either side of the needle 20. As shown, by placing the insides of the cutter blades 15 very close to the outside surface of the needle 20, a continuous incision can be made extending form the incision cut by needle 20.

The cutter blades 15 are in principle disposed in the cursor housing 32 symmetrically about the needle 20 when the cursor 30 is mounted on the needle 20. As shown in FIG. 2, the cutting edges 16 of cutting blades 15 make an angle of at most 45 degrees and preferably up to 30 degrees with the needle. In the illustrated example the angle is about 20 degrees.

Generally, as in the illustrated embodiment, the cursor housing 32 is substantially flat and the cutter blades 15 lie flat in the substantially flat housing 32, as can be seen in the cross-section of FIG. 3.

The needle 20 can be pre-assembled with the cursor housing 32, forming a pre-assembled unit with the cursor housing 32 slidably mounted on the needle 20. Alternatively, the needle 20 and cursor housing 32 are provided separately as a set of parts that can be assembled in preparation for use.

Using a hollow needle 20, the cutter 10 can be combined with a flexible wire or tube that can be passed through the needle 20. Typically, the hollow needle has an outer diameter in the range 1 mm to 6 mm and the cursor housing 32 and cutter blade assembly has a through-passage of corresponding dimensions for slidably receiving the needle 20.

As shown in FIG. 2, the cutter blades 15 can be assembled with a central cylindrical piece 42 or a part-cylindrical piece to form a cutter blade assembly having one cutter blade 15 on either side of the central cylindrical piece 42, like a delta wing.

The described cutter is convenient to use and enables a surgeon to make an incision with great precision with little or no risk of accident. The described cutter is also easy to manufacture and can be mass-produced as a disposable.

1. A cutter (10) for use in surgery, comprising:
a needle (20) having a cutting end (21); and
a cursor (30) slidably mountable on or mounted on the needle (20), the cursor comprising:
a cursor housing (32) slidably mountable on or mounted on the needle (20) with the cursor housing (32) having an open end facing the needle cutting end; and
a pair of flat cutter blades (15) mounted in the cursor housing (32) on either side of the needle (20), the cutter blades having cutting edges (16) facing the open end of the cursor housing (32), the cutter blades (15) being mounted for movement together relative to the cursor housing between a first stored position wherein the cutter blades (15) are enclosed in the cursor housing (32) and a second projecting position wherein the cutting edges (16) of the cutter blades (15) project from the open end of the cursor housing (32), the cutter blades (15) being movable from the first to the second position against the action of biasing means (38) that act to return the cutter blades to the first position;
the cutter (10) being operable for making a cut or an incision in a surface (40) by bringing the needle cutting end (21) into contact with the surface to be cut, sliding the cursor (30) along the needle (20) up to adjacent the surface to be cut, and moving the cutter blades (15) to the projecting second position against the action of the biasing means (38) to bring the cutting edges (16) to a cutting position for making a cut or incision in the surface on either side of the needle.

2. The cutter of claim 1 wherein the cutter blades (15) are disposed in the cursor housing (32) symmetrically about the needle (20) when the cursor (30) is mounted on the needle.

3. The cutter of claim 2 wherein the cutting edges (16) of the cutting blades (15) make an angle of at most 45 degrees and preferably up to 30 degrees with the needle (20).

4. The cutter of any preceding claim wherein the cursor housing (32) is substantially flat and the cutter blades (15) lie flat in the substantially flat housing.

5. The cutter of any preceding claim wherein the cursor housing (32) has, at an end of the cursor (30) opposite said open end, two laterally-projecting finger grips (39) for facilitating manipulation of the cursor housing.

6. The cutter of any preceding claim comprising an actuator (12) for facilitating movement of the cutter blades (15) from the first to the second position.

7. The cutter of claim 6 wherein said actuator (12) is a press button.

8. The cutter of any preceding claim wherein the needle (20) is pre-assembled with the cursor housing (32), forming a pre-assembled unit in which the cursor housing (32) is slidably mounted on the needle (20).

9. The cutter of any one of claims 1 to 7 wherein the needle (20) and cursor housing (32) are provided separately as a set of parts that can be assembled in preparation for use.

10. The cutter of any preceding claim wherein the needle (20) is hollow.

11. The cutter of claim 10 in combination with a flexible wire or tube that can be passed through the needle (20).

12. The cutter of any preceding claim wherein the needle (20) has an outer diameter in the range 1 mm to 6 mm and the cursor housing (32) has a through-passage of corresponding dimensions for slidably receiving the needle (20).

13. The cutter of any preceding claim wherein the cutter blades (15) are assembled with a central cylindrical piece (42) or part-cylindrical piece to form a cutter blade unit having one cutter blade (15) on either side of the central cylindrical or part-cylindrical piece (42).

14. The cutter of any preceding claim wherein the cutter blades (15) are mounted on a support (14) slidably mounted in the cursor housing (32).

15. A cursor and cutting blade assembly of a cutter according to any preceding claim, which cutter is adapted to be used in combination with a needle (20) having a cutting end, said cursor and cutting blade assembly comprising:
a cursor housing (32) slidably mountable on an associated needle (20), the cursor housing (32) when mounted on the needle having an open end facing the needle cutting end; and
a pair of flat cutter blades (15) mounted in the cursor housing (32) on either side of the needle (20) when mounted, the cutter blades (15) having cutting edges (16) facing the open end of the cursor housing (32), the cutter blades (15) being mounted for movement together relative to the cursor housing (32) between a first stored position wherein the cutter blades (15) are enclosed in the cursor housing (32) and a second projecting position wherein the cutting edges (16) of the cutter blades (15) project from the open end of the cursor housing (32), the cutter blades (15) being movable from the first to the second position against the action of biasing means (38) that act to return the cutter blades (15) to the first position.

16. The cursor and cutting blade assembly of claim 15 wherein the cursor housing (32) has a central through-passage passing between the cutting blades, for slidably receiving a needle (20) of corresponding dimensions.
